SEQUENCE LISTING (34 sequences)

SEQ ID NO: 1 - DNA, 20 bp, Artificial Sequence
  primer ZEB1 Fw
ggcagagaatgagggagaag

SEQ ID NO: 2 - DNA, 23 bp, Artificial Sequence
  primer ZEB1 Rv
cttcagacacttgctcactactc

SEQ ID NO: 3 - DNA, 17 bp, Artificial Sequence
  primer CLDN1-s
ctgccttctgggaggtg

SEQ ID NO: 4 - DNA, 16 bp, Artificial Sequence
  primer CLDN1-as
cgctggaaggtgcagg

SEQ ID NO: 5 - DNA, 21 bp, Artificial Sequence
  primer CALML3-F
ggagaagctgagtgacgagga

SEQ ID NO: 6 - DNA, 20 bp, Artificial Sequence
  primer CALML3-R
acaccagcacacggacaaac

SEQ ID NO: 7 - DNA, 18 bp, Artificial Sequence
  primer BGN-F
atccacgacaaccgcatc

SEQ ID NO: 8 - DNA, 21 bp, Artificial Sequence
  primer BGN-R
ccaggttcaaagccactgttc

SEQ ID NO: 9 - DNA, 19 bp, Artificial Sequence
  primer CLCA2-F
cccagcccactctattcca

SEQ ID NO: 10 - DNA, 22 bp, Artificial Sequence
  primer CLCA2-R
gctcctcctcatttctgcctac

SEQ ID NO: 11 - DNA, 18 bp, Artificial Sequence
  primer CSTA-F
cgccactccagaaatcca

SEQ ID NO: 12 - DNA, 21 bp, Artificial Sequence
  primer CSTA-R
ctgcacagcttccaattttcc

SEQ ID NO: 13 - DNA, 23 bp, Artificial Sequence
  primer ALDH1A1-F
gccataacaatctcctctgctct

SEQ ID NO: 14 - DNA, 21 bp, Artificial Sequence
  primer ALDH1A1-R
cccagttctcttccatttcca

SEQ ID NO: 15 - DNA, 22 bp, Artificial Sequence
  primer NGFR-F
tccctgtctattgctccatcct

SEQ ID NO: 16 - DNA, 19 bp, Artificial Sequence
  primer NGFR-R
gctgttggctccttgcttg

SEQ ID NO: 17 - DNA, 25 bp, Artificial Sequence
  primer S100A8-F
gagaccgagtgtcctcagtatatca

SEQ ID NO: 18 - DNA, 20 bp, Artificial Sequence
  primer S100A8-R
cacgcccatctttatcacca

SEQ ID NO: 19 - DNA, 21 bp, Artificial Sequence
  primer ELN-F
gcagctaaatacggtgctgct

SEQ ID NO: 20 - DNA, 20 bp, Artificial Sequence
  primer ELN-R
cctgggaaaatgggagacaa

SEQ ID NO: 21 - DNA, 21 bp, Artificial Sequence
  primer SNURF-F
atcctgcaagatggccgaatc

SEQ ID NO: 22 - DNA, 22 bp, Artificial Sequence
  primer SNURF-R
tggttgcttcgcattctttggc

SEQ ID NO: 23 - DNA, 22 bp, Artificial Sequence
  primer LGALS7/7B-F
tgccagcaggttccatgtaaac

SEQ ID NO: 24 - DNA, 22 bp, Artificial Sequence
  primer LGALS7/7B-R
ttgctccttgctgttgaagacc

SEQ ID NO: 25 - DNA, 1417 bp, Homo sapiens
  misc_feature: CALML3
agggcaggggcgtctgccaatgatgggggaggactctgctgcttcttaagctccagcgtctcaagccagggcgagacagcccgccggccgcccggatctccacctgccaccccagagctgggacagcagccgggctgcggcactgggagggagaccccacagtggcctcttctgccacccacgcccccacccctggcatggccgaccagctgactgaggagcaggtcacagaattcaaggaggccttctccctgtttgacaaggatggggacggctgcatcaccacccgcgagctgggcacggtcatgcggtccctgggccagaaccccacggaggccgagctgcgggacatgatgagtgagatcgaccgggacggcaacggcaccgtggacttccccgagttcctgggcatgatggccaggaagatgaaggacacggacaacgaggaggagatccgcgaggccttccgcgtgttcgacaaggacggcaacggcttcgtcagcgccgccgagctgcgacacgtcatgacccggctgggggagaagctgagtgacgaggaggtggacgagatgatccgggccgcggacacggacggagacggacaggtgaactacgaggagtttgtccgtgtgctggtgtccaagtgaggccggcgcccaccatgctcctgggcgcccacgcggcccacagggcaagaacccggggcctcccgcctcctcccccatccccctgcctcccctgggcactgtggcttcctcctgcgcctggttgattcagcccacctctctgcatcccgcttcccgcgtctcttctctgcactcctgccgaccttcccacctgctcgtctgaatgacacggaacgctcccactgcaggcaaaccgtgacgccctccccactcgggagaagcagagctgaccttaggaccgagcaccagggcaggttgcgctgactctgcggccctccaggacggacaccgggtgaccccttagggcacccaggcaagatccctaagaggcacccaatgcccaggccaggggggctgcagccctcagcccccgccaggattcccgcaggctcctggactggaagctccctccgcggtcggattctggagggtgggaggcatcttggcctgcagtaagcggtgctgacggggactctggccacagaggtcaggcctcctgaaaacagcactgccttccgcgctgccccagcttgccccattccttgtccgccaacccaccgtgattcatcttctgaagctgggagtgaaactgggtcagctgtaacctgttcctattcatctggaaggagggaggcttggatgagcaggggatgagagctgcagggaaataaatgagatattcgtccttatttcaaaaaaaaaaaaaaaaaaaaaaaaaaaaaaaaaaaaaa

SEQ ID NO: 26 - DNA, 2470 bp, Homo sapiens
  misc_feature: BGN
cctttcctccctccccgccctctccccgctgtcccctccccgtcggcccgcctgcccagcctttagcctcccgcccgccgcctctgtctccctctctccacaaactgcccaggagtgagtagctgctttcggtccgccggacacaccggacagatagacgtgcggacggcccaccaccccagcccgccaactagtcagcctgcgcctggcgcctcccctctccaggtccatccgccatgtggcccctgtggcgcctcgtgtctctgctggccctgagccaggccctgccctttgagcagagaggcttctgggacttcaccctggacgatgggccattcatgatgaacgatgaggaagcttcgggcgctgacacctcgggcgtcctggacccggactctgtcacacccacctacagcgccatgtgtcctttcggctgccactgccacctgcgggtggttcagtgctccgacctgggtctgaagtctgtgcccaaagagatctcccctgacaccacgctgctggacctgcagaacaacgacatctccgagctccgcaaggatgacttcaagggtctccagcacctctacgccctcgtcctggtgaacaacaagatctccaagatccatgagaaggccttcagcccactgcggaagctgcagaagctctacatctccaagaaccacctggtggagatcccgcccaacctacccagctccctggtggagctccgcatccacgacaaccgcatccgcaaggtgcccaagggagtgttcagcgggctccggaacatgaactgcatcgagatgggcgggaacccactggagaacagtggctttgaacctggagccttcgatggcctgaagctcaactacctgcgcatctcagaggccaagctgactggcatccccaaagacctccctgagaccctgaatgaactccacctagaccacaacaaaatccaggccatcgaactggaggacctgcttcgctactccaagctgtacaggctgggcctaggccacaaccagatcaggatgatcgagaacgggagcctgagcttcctgcccaccctccgggagctccacttggacaacaacaagttggccagggtgccctcagggctcccagacctcaagctcctccaggtggtctatctgcactccaacaacatcaccaaagtgggtgtcaacgacttctgtcccatgggcttcggggtgaagcgggcctactacaacggcatcagcctcttcaacaaccccgtgccctactgggaggtgcagccggccactttccgctgcgtcactgaccgcctggccatccagtttggcaactacaaaaagtagaggcagctgcagccaccgcggggcctcagtgggggtctctggggaacacagccagacatcctgatggggaggcagagccaggaagctaagccagggcccagctgcgtccaacccagccccccacctcgggtccctgaccccagctcgatgccccatcaccgcctctccctggctcccaagggtgcaggtgggcgcaaggcccggcccccatcacatgttcccttggcctcagagctgcccctgctctcccaccacagccacccagaggcaccccatgaagcttttttctcgttcactcccaaacccaagtgtccaaggctccagtcctaggagaacagtccctgggtcagcagccaggaggcggtccataagaatggggacagtgggctctgccagggctgccgcacctgtccagacacacatgttctgttcctcctcctcatgcatttccagcctttcaaccctccccgactctgcggctcccctcagcccccttgcaagttcatggcctgtccctcccagacccctgctccactggcccttcgaccagtcctcccttctgttctctctttccccgtccttcctctctctctctctctctctctctctctctttctgtgtgtgtgtgtgtgtgtgtgtgtgtgtgtgtgtgtgtgtgtgtcttgtgcttcctcagacctttctcgcttctgagcttggtggcctgttccctccatctctccgaacctggcttcgcctgtccctttcactccacaccctctggccttctgccttgagctgggactgctttctgtctgtccggcctgcacccagcccctgcccacaaaaccccagggacagcagtctccccagcctgccctgctcaggccttgcccccaaacctgtactgtcccggaggaggttgggaggtggaggcccagcatcccgcgcagatgacaccatcaaccgccagagtcccagacaccggttttcctagaagcccctcacccccactggcccactggtggctaggtctccccttatccttctggtccagcgcaaggaggggctgcttctgaggtcggtggctgtctttccattaaagaaacaccgtgcaacgtggctccgtgaaaaaaaaaaaaaaaaaa

SEQ ID NO: 27 - DNA, 4043 bp, Homo sapiens
  misc_feature: CLCA2
tcccagatggatccaccccagacttttcaaagaagacacctccttcatcttgtgttctaaaaccttgcaagttcaggaagaaaccatctgcatccatattgaaaacctgacacaatgtatgcagcaggctcagtgtgagtgaactggaggcttctctacaacatgacccaaaggagcattgcaggtcctatttgcaacctgaagtttgtgactctcctggttgccttaagttcagaactcccattcctgggagctggagtacagcttcaagacaatgggtataatggattgctcattgcaattaatcctcaggtacctgagaatcagaacctcatctcaaacattaaggaaatgataactgaagcttcattttacctatttaatgctaccaagagaagagtatttttcagaaatataaagattttaatacctgccacatggaaagctaataataacagcaaaataaaacaagaatcatatgaaaaggcaaatgtcatagtgactgactggtatggggcacatggagatgatccatacaccctacaatacagagggtgtggaaaagagggaaaatacattcatttcacacctaatttcctactgaatgataacttaacagctggctacggatcacgaggccgagtgtttgtccatgaatgggcccacctccgttggggtgtgttcgatgagtataacaatgacaaacctttctacataaatgggcaaaatcaaattaaagtgacaaggtgttcatctgacatcacaggcatttttgtgtgtgaaaaaggtccttgcccccaagaaaactgtattattagtaagctttttaaagaaggatgcacctttatctacaatagcacccaaaatgcaactgcatcaataatgttcatgcaaagtttatcttctgtggttgaattttgtaatgcaagtacccacaaccaagaagcaccaaacctacagaaccagatgtgcagcctcagaagtgcatgggatgtaatcacagactctgctgactttcaccacagctttcccatgaatgggactgagcttccacctcctcccacattctcgcttgtacaggctggtgacaaagtggtctgtttagtgctggatgtgtccagcaagatggcagaggctgacagactccttcaactacaacaagccgcagaattttatttgatgcagattgttgaaattcataccttcgtgggcattgccagtttcgacagcaaaggagagatcagagcccagctacaccaaattaacagcaatgatgatcgaaagttgctggtttcatatctgcccaccactgtatcagctaaaacagacatcagcatttgttcagggcttaagaaaggatttgaggtggttgaaaaactgaatggaaaagcttatggctctgtgatgatattagtgaccagcggagatgataagcttcttggcaattgcttacccactgtgctcagcagtggttcaacaattcactccattgccctgggttcatctgcagccccaaatctggaggaattatcacgtcttacaggaggtttaaagttctttgttccagatatatcaaactccaatagcatgattgatgctttcagtagaatttcctctggaactggagacattttccagcaacatattcagcttgaaagtacaggtgaaaatgtcaaacctcaccatcaattgaaaaacacagtgactgtggataatactgtgggcaacgacactatgtttctagttacgtggcaggccagtggtcctcctgagattatattatttgatcctgatggacgaaaatactacacaaataattttatcaccaatctaacttttcggacagctagtctttggattccaggaacagctaagcctgggcactggacttacaccctgaacaatacccatcattctctgcaagccctgaaagtgacagtgacctctcgcgcctccaactcagctgtgcccccagccactgtggaagcctttgtggaaagagacagcctccattttcctcatcctgtgatgatttatgccaatgtgaaacagggattttatcccattcttaatgccactgtcactgccacagttgagccagagactggagatcctgttacgctgagactccttgatgatggagcaggtgctgatgttataaaaaatgatggaatttactcgaggtattttttctcctttgctgcaaatggtagatatagcttgaaagtgcatgtcaatcactctcccagcataagcaccccagcccactctattccagggagtcatgctatgtatgtaccaggttacacagcaaacggtaatattcagatgaatgctccaaggaaatcagtaggcagaaatgaggaggagcgaaagtggggctttagccgagtcagctcaggaggctccttttcagtgctgggagttccagctggcccccaccctgatgtgtttccaccatgcaaaattattgacctggaagctgtaaaagtagaagaggaattgaccctatcttggacagcacctggagaagactttgatcagggccaggctacaagctatgaaataagaatgagtaaaagtctacagaatatccaagatgactttaacaatgctattttagtaaatacatcaaagcgaaatcctcagcaagctggcatcagggagatatttacgttctcaccccaaatttccacgaatggacctgaacatcagccaaatggagaaacacatgaaagccacagaatttatgttgcaatacgagcaatggataggaactccttacagtctgctgtatctaacattgcccaggcgcctctgtttattccccccaattctgatcctgtacctgccagagattatcttatattgaaaggagttttaacagcaatgggtttgataggaatcatttgccttattatagttgtgacacatcatactttaagcaggaaaaagagagcagacaagaaagagaatggaacaaaattattataaataaatatccaaagtgtcttccttcttagatataagacccatggccttcgactacaaaaacatactaacaaagtcaaattaacatcaaaactgtattaaaatgcattgagtttttgtacaatacagataagatttttacatggtagatcaacaaattctttttgggggtagattagaaaacccttacactttggctatgaacaaataataaaaattattctttaaagtaatgtctttaaaggcaaagggaagggtaaagtcggaccagtgtcaaggaaagtttgttttattgaggtggaaaaatagccccaagcagagaaaaggagggtaggtctgcattataactgtctgtgtgaagcaatcatttagttactttgattaatttttcttttctccttatctgtgcagaacaggttgcttgtttacaactgaagatcatgctatattttatatatgaagcccctaatgcaaagctctttacctcttgctattttgttatatatattacagatgaaatctcactgctaatgctcagagatcttttttcactgtaagaggtaacctttaacaatatgggtattacctttgtctcttcataccggttttatgacaaaggtctattgaatttatttgtttgtaagtttctactcccatcaaagcagctttctaagttattgccttggttattatggatgatagttatagcccttataatgccttaactaaggaagaaaagatgttattctgagtttgttttaatacatatatgaacatatagttttattcaattaaaccaaagaagaggtcagcagggagatactaacctttggaaatgattagctggctctgttttttggttaaataagagtctttaatcctttctccatcaagagttacttaccaagggcaggggaagggggatatagaggtcacaaggaaataaaaatcatctttcatctttaattttactccttcctcttatttttttaaaagattatcgaacaataaaatcatttgcctttttaattaaaaaaaaaaaaaaaaaaaaa

SEQ ID NO: 28 - DNA, 838 bp, Homo sapiens
  misc_feature: CSTA
tgctgtttgtggaaaataaagcattctataggcggagctagtgaacgcctcttttaaaacacgagtctccacacttccctgttcactttggttccagcatcctgtccagcaaagaagcaatcagccaaaatgatacctggaggcttatctgaggccaaacccgccactccagaaatccaggagattgttgataaggttaaaccacagcttgaagaaaaaacaaatgagacttacggaaaattggaagctgtgcagtataaaactcaagttgttgctggaacaaattactacattaaggtacgagcaggtgataataaatatatgcacttgaaagtattcaaaagtcttcccggacaaaatgaggacttggtacttactggataccaggttgacaaaaacaaggatgacgagctgacgggcttttagcagcatgtacccaaagtgttctgattccttcaactggctactgagtcatgatccttgctgataaatataaccatcaataaagaagcattcttttccaaagaaattatttcttcaattatttctcatttattgtattaagcagaaattaccttttctttctcaaaatcagtgttattgctttagagtataaactccatataaattgatggcaattggaaatcttataaaaactagtcaagcctaatgcaactggctaaaggatagtaccaccctcacccccaccataggcaggctggatcgtggactatcaattcaccagcctccttgttccctgtggctgctgataacccaacattccatctctaccctcatacttcaaaattaaatcaagtattttacaaaaaaaaaaaaa

SEQ ID NO: 29 - DNA, 2378 bp, Homo sapiens
  misc_feature: ALDH1A1
gctgcatacacttatcacaggtttcggctttgtaaattaattcatctgcaaatagtgcactgtctccaggtacaaattcgatgctggagcactggtttcttaaggatttaagtttaaagtcaaaggcttcctgccctaggtgttacaaataagtagtgtcgttttctttttttgctctgagtttgttcatccaatcgtatccgagtatgcaaataaactttagcccgtgcagataaaaaaggaacaaataaagccaagtgctctatcagaaccaaattgctgagccagtcacctgtgttccaggagccgaatcagaaatgtcatcctcaggcacgccagacttacctgtcctactcaccgatttgaagattcaatatactaagatcttcataaacaatgaatggcatgattcagtgagtggcaagaaatttcctgtctttaatcctgcaactgaggaggagctctgccaggtagaagaaggagataaggaggatgttgacaaggcagtgaaggccgcaagacaggcttttcagattggatccccgtggcgtactatggatgcttccgagagggggcgactattatacaagttggctgatttaatcgaaagagatcgtctgctgctggcgacaatggagtcaatgaatggtggaaaactctattccaatgcatatctgaatgatttagcaggctgcatcaaaacattgcgctactgtgcaggttgggctgacaagatccagggccgtacaataccaattgatggaaatttttttacatatacaagacatgaacctattggtgtatgtggccaaatcattccttggaatttcccgttggttatgctcatttggaagatagggcctgcactgagctgtggaaacacagtggttgtcaaaccagcagagcaaactcctctcactgctctccacgtggcatctttaataaaagaggcagggtttcctcctggagtagtgaatattgttcctggttatgggcctacagcaggggcagccatttcttctcacatggatatagacaaagtagccttcacaggatcaacagaggttggcaagttgatcaaagaagctgccgggaaaagcaatctgaagagggtgaccctggagcttggaggaaagagcccttgcattgtgttagctgatgccgacttggacaatgctgttgaatttgcacaccatggggtattctaccaccagggccagtgttgtatagccgcatccaggatttttgtggaagaatcaatttatgatgagtttgttcgaaggagtgttgagcgggctaagaagtatatccttggaaatcctctgaccccaggagtcactcaaggccctcagattgacaaggaacaatatgataaaatacttgacctcattgagagtgggaagaaagaaggggccaaactggaatgtggaggaggcccgtgggggaataaaggctactttgtccagcccacagtgttctctaatgttacagatgagatgcgcattgccaaagaggagatttttggaccagtgcagcaaatcatgaagtttaaatctttagatgacgtgatcaaaagagcaaacaatactttctatggcttatcagcaggagtgtttaccaaagacattgataaagccataacaatctcctctgctctgcaggcaggaacagtgtgggtgaattgctatggcgtggtaagtgcccagtgcccctttggtggattcaagatgtctggaaatggaagagaactgggagagtacggtttccatgaatatacagaggtcaaaacagtcacagtgaaaatctctcagaagaactcataaagaaaatacaagagtggagagaagctcttcaatagctaagcatctccttacagtcactaatatagtagattttaaagacaaaatttttcttttcttgatttttttaaacataagctaaatcatattagtattaatactacccatagaaaacttgacatgtagcttcttctgaaagaattatttgccttctgaaatgtgacccccaagtcctatcctaaataaaaaaagacaaattcggatgtatgatctctctagctttgtcatagttatgtgattttcctttgtagctacttttgcaggataataattttatagaaaaggaacagttgcatttagcttctttcccttagtgactcttgaagtacttaacatacacgttaactgcagagtaaattgctctgttcccagtagttataaagtccttggactgttttgaaaagtttcctaggatgtcatgtctgcttgtcaaaagaaataatccctgtaatatttagctgtaaactgaatataaagcttaataaaaacaaccttgcatgattcttgttaaaaaaaaaa

SEQ ID NO: 30 - DNA, 3420 bp, Homo sapiens
  misc_feature: NGFR
agagcgagccgagccgcggccagctccggcgggcagggggggcgctggagcgcagcgcagcgcagccccatcagtccgcaaagcggaccgagctggaagtcgagcgctgccgcgggaggcgggcgatgggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggccttgtggcctacatagccttcaagaggtggaacagctgcaagcagaacaagcaaggagccaacagccggccagtgaaccagacgcccccaccagagggagaaaaactccacagcgacagtggcatctccgtggacagccagagcctgcatgaccagcagccccacacgcagacagcctcgggccaggccctcaagggtgacggaggcctctacagcagcctgcccccagccaagcgggaggaggtggagaagcttctcaacggctctgcgggggacacctggcggcacctggcgggcgagctgggctaccagcccgagcacatagactcctttacccatgaggcctgccccgttcgcgccctgcttgcaagctgggccacccaggacagcgccacactggacgccctcctggccgccctgcgccgcatccagcgagccgacctcgtggagagtctgtgcagtgagtccactgccacatccccggtgtgagcccaaccggggagcccccgccccgccccacattccgacaaccgatgctccagccaacccctgtggagcccgcacccccaccctttggggggggcccgcctggcagaactgagctcctctgggcaggacctcagagtccaggccccaaaaccacagccctgtcagtgcagcccgtgtggccccttcacttctgaccacacttcctgtccagagagagaagtgcccctgctgcctccccaaccctgcccctgccccgtcaccatctcaggccacctgcccccttctcccacactgctaggtgggccagcccctcccaccacagcaggtgtcatatatggggggccaacaccagggatggtactagggggaagtgacaaggccccagagactcagagggaggaatcgaggaaccagagccatggactctacactgtgaacttggggaacaagggtggcatcccagtggcctcaaccctccctcagcccctcttgccccccaccccagcctaagatgaagaggatcggaggcttgtcagagctgggaggggttttcgaagctcagcccacccccctcattttggatataggtcagtgaggcccagggagaggccatgattcgcccaaagccagacagcaacggggaggccaagtgcaggctggcaccgccttctctaaatgaggggcctcaggtttgcctgagggcgaggggagggtggcaggtgaccttctgggaaatggcttgaagccaagtcagctttgccttccacgctgtctccagacccccaccccttccccactgcctgcccacccgtggagatgggatgcttgcctagggcctggtccatgatggagtcaggtttggggttcgtggaaagggtgctgcttccctctgcctgtccctctcaggcatgcctgtgtgacatcagtggcatggctccagtctgctgccctccatcccgacatggacccggagctaacactggcccctagaatcagcctaggggtcagggaccaaggacccctcaccttgcaacacacagacacacgcacacacacacacaggaggagaaatctcacttttctccatgagttttttctcttgggctgagactggatactgcccggggcagctgccagagaagcatcggagggaattgaggtctgctcggccgtcttcactcgcccccgggtttggcgggccaaggactgccgaccgaggctggagctggcgtctgtcttcaagggcttacacgtggaggaatgctcccccatcctccccttccctgcaaacatggggttggctgggcccagaaggttgtgatgaagaaaagtgggccagtgtgggaatgcggcaagaaggaattgacttcgactgtgacctgtggggatttctcccagctctagacaaccctgcaaaggactgttttttcctgagcttggccagaagggggccatgaggcctcagtggactttccaccccctccctggcctgttctgttttgcctgaagttggagtgagtgtggctcccctctatttagcatgacaagccccaggcaggctgtgcgctgacaaccaccgctccccagcccagggttcccccagccctgtggaagggactaggagcactgtagtaaatggcaattctttgacctcaacctgtgatgaggggaggaaactcacctgctggcccctcacctgggcacctggggagtgggacagagtctgggtgtatttattttcctccccagcaggtggggagggggtttgggggcttgcaagtatgttttagcatgtgtttggttctggggcccctttttactccccttgagctgagatggaacccttttggcccccgagctgggggccatgagctccagacccccagcaaccctcctatcacctcccctccttgcctcctgtgtaatcatttcttgggccctcctgaaacttacacacaaaacgttaagtgatgaacattaaatagcaaagaaagaaaaatagtacaaagag

SEQ ID NO: 31 - DNA, 532 bp, Homo sapiens
  misc_feature: S100A8
gagaaaccagagactgtagcaactctggcagggagaagctgtctctgatggcctgaagctgtgggcagctggccaagcctaaccgctataaaaaggagctgcctctcagccctgcatgtctcttgtcagctgtctttcagaagacctggtggggcaagtccgtgggcatcatgttgaccgagctggagaaagccttgaactctatcatcgacgtctaccacaagtactccctgataaaggggaatttccatgccgtctacagggatgacctgaagaaattgctagagaccgagtgtcctcagtatatcaggaaaaagggtgcagacgtctggttcaaagagttggatatcaacactgatggtgcagttaacttccaggagttcctcattctggtgataaagatgggcgtggcagcccacaaaaaaagccatgaagaaagccacaaagagtagctgagttactgggcccagaggctgggcccctggacatgtacctgcagaataataaagtcatcaatacctcaaaaaaaaaa

SEQ ID NO: 32 - DNA, 3789 bp, Homo sapiens
  misc_feature: ELN
cctcccttctgcttcctctcccgagggctgtcctggcagaggcccccctcgctctttctggcgggaacagggccagcagcgaaagaacagtcgcagagggaaagcgggaaagagatgggggaaagtgtgtgtgtgtgagtgtgtgcttgtgtgcatgtgtgtgcgtgtgtgtgtcaaggaaaaaagctcgcagtccagcagcccgggcctgggaggcttgtgagccgggcctttcgtaattgtcccctccccgcggccccctcccccaggcctcccccctctcccgccctcccgcccgccctctctccctccctctttccctcacagccgacgaggcaacaattaggctttggggataaaacgaggtgcggagagcgggctggggcatttctccccgagatggcgggtctgacggcggcggccccgcggcccggagtcctcctgctcctgctgtccatcctccacccctctcggcctggaggggtccctggggccattcctggtggagttcctggaggagtcttttatccaggggctggtctcggagcccttggaggaggagcgctggggcctggaggcaaacctcttaagccagttcccggagggcttgcgggtgctggccttggggcagggctcggcgccttccccgcagttacctttccgggggctctggtgcctggtggagtggctgacgctgctgcagcctataaagctgctaaggctggcgctgggcttggtggtgtcccaggagttggtggcttaggagtgtctgcaggtgcggtggttcctcagcctggagccggagtgaagcctgggaaagtgccgggtgtggggctgccaggtgtatacccaggtggcgtgctcccaggagctcggttccccggtgtgggggtgctccctggagttcccactggagcaggagttaagcccaaggctccaggtgtaggtggagcttttgctggaatcccaggagttggaccctttgggggaccgcaacctggagtcccactggggtatcccatcaaggcccccaagctgcctggtggctatggactgccctacaccacagggaaactgccctatggctatgggcccggaggagtggctggtgcagcgggcaaggctggttacccaacagggacaggggttggcccccaggcagcagcagcagcggcagctaaagcagcagcaaagttcggtgctggagcagccggagtcctccctggtgttggaggggctggtgttcctggcgtgcctggggcaattcctggaattggaggcatcgcaggcgttgggactccagctgcagctgcagctgcagcagcagccgctaaggcagccaagtatggagctgctgcaggcttagtgcctggtgggccaggctttggcccgggagtagttggtgtcccaggagctggcgttccaggtgttggtgtcccaggagctgggattccagttgtcccaggtgctgggatcccaggtgctgcggttccaggggttgtgtcaccagaagcagctgctaaggcagctgcaaaggcagccaaatacggggccaggcccggagtcggagttggaggcattcctacttacggggttggagctgggggctttcccggctttggtgtcggagtcggaggtatccctggagtcgcaggtgtccctggtgtcggaggtgttcccggagtcggaggtgtcccgggagttggcatttcccccgaagctcaggcagcagctgccgccaaggctgccaagtacggagtggggaccccagcagctgcagctgctaaagcagccgccaaagccgcccagtttgggttagttcctggtgtcggcgtggctcctggagttggcgtggctcctggtgtcggtgtggctcctggagttggcttggctcctggagttggcgtggctcctggagttggtgtggctcctggcgttggcgtggctcccggcattggccctggtggagttgcagctgcagcaaaatccgctgccaaggtggctgccaaagcccagctccgagctgcagctgggcttggtgctggcatccctggacttggagttggtgtcggcgtccctggacttggagttggtgctggtgttcctggacttggagttggtgctggtgttcctggcttcggggcagtacctggagccctggctgccgctaaagcagccaaatatggagcagcagtgcctggggtccttggagggctcggggctctcggtggagtaggcatcccaggcggtgtggtgggagccggacccgccgccgccgctgccgcagccaaagctgctgccaaagccgcccagtttggcctagtgggagccgctgggctcggaggactcggagtcggagggcttggagttccaggtgttgggggccttggaggtatacctccagctgcagccgctaaagcagctaaatacggtgctgctggccttggaggtgtcctagggggtgccgggcagttcccacttggaggagtggcagcaagacctggcttcggattgtctcccattttcccaggtggggcctgcctggggaaagcttgtggccggaagagaaaatgagcttcctaggacccctgactcacgacctcatcaacgttggtgctactgcttggtggagaatgtaaaccctttgtaaccccatcccatgcccctccgactccccaccccaggagggaacgggcaggccgggcggccttgcagatccacagggcaaggaaacaagaggggagcggccaagtgccccgaccaggaggccccctacttcagaggcaagggccatgtggtcctggccccccaccccatcccttcccacctaggagctccccctccacacagcctccatctccaggggaacttggtgctacacgctggtgctcttatcttcctggggggagggaggagggaagggtggcccctcggggaaccccctacctggggctcctctaaagatggtgcagacacttcctgggcagtcccagctccccctgcccaccaggacccaccgttggctgccatccagttggtacccaagcacctgaagcctcaaagctggattcgctctagcatccctcctctcctgggtccacttggccgtctcctccccaccgatcgctgttccccacatctggggcgcttttgggttggaaaaccaccccacactgggaatagccaccttgcccttgtagaatccatccgcccatccgtccattcatccatcggtccgtccatccatgtccccagttgaccgcccggcaccactagctggctgggtgcacccaccatcaacctggttgacctgtcatggccgcctgtgccctgcctccacccccatcctacactcccccagggcgtgcggggctgtgcagactggggtgccaggcatctcctccccacccggggtgtccccacatgcagtactgtataccccccatccctccctcggtccactgaacttcagagcagttcccattcctgccccgcccatctttttgtgtctcgctgtgatagatcaataaatattttattttttgtcctggatatttggggattatttttgattgttgatattctcttttggttttattgttgtggttcattgaaaaaaaaagataatttttttttctgatccggggagctgtatccccagtagaaaaaacattttaatcactctaatataactctggatgaaacacacctttttttttaataagaaaagagaattaactgcttcagaaatgactaataaatgaaaaacctttaaaggaaaaaaaaaaa

SEQ ID NO: 33 - DNA, 1575 bp, Homo sapiens
  misc_feature: SNURF
aaacaagcacgcctgcgcggccgcagaggcaggctggcgcgcatgctcaggcggggatgtgtgcgaagcctgccgctgctgcagcgagtctggcgcagagtggagcggccgccggagatgcctgacgcatctgtctgaggagcggtcagtgacgcgatggagcgggcaagggatcgcttacacctgagacgaactacagaacagcacgtaccagaggtggaagtccaagtcaaacgcagaaggactgcctcactgagcaaccaagagtgtcagttgtacccgaggcgttctcagcagcagcaagtacctgtggtggatttccaggctgaactgaggcaggcattcttagctgagacaccaagaggtggttaaagccatattggagtagcgaggaatctgattccaagcaaaaaccaggctccatctactctttgaagcttctgcccagcttgcattgtttctaggagaacctgcgtcatacctttatctatagccttcccctaggtcttcagaagcatcaagttttaactgtggacattggatttggtggaacagcaatcatgactgttggcaagagtagcaagatgctgcagcacattgactatagaatgagatgtatcctgcaagatggccgaatcttcattggcacctttaaggcttttgacaagcatatgaatttgatcctctgtgattgtgatgagttcagaaagatcaagccaaagaatgcgaagcaaccagagcgtgaagaaaagcgggttttgggtctggtgttgctgcgtggggagaacttggtatccatgactgtggaggggccaccccccaaagatactggcattgctcgggtaccacttgctggagctgctggaggccctggggttggtagggcagctggtagaggagtaccagctggtgtgccaattccccaggcccctgctggattggcaggccctgtccgaggagttgggggaccatcccagcaggtaatgactccacagggaagaggcactgtagcagctgctgctgttgctgcgactgccagtattgctggagccccaacacagtacccaccaggacggggcactccgcccccacccgtcggcagagcaaccccacctccaggcattatggctcctccacctggtatgagaccacccatgggcccaccaattgggcttccccctgctcgagggacgccaataggcatgccgcctccgggaatgagaccccctccaccaggcattagaggtccacctcccccaggaatgcgtccaccaagaccttagcatactgttgatccatctcagtcactttttcccctgcaatgcgtcttgtgaaattgtgtagagtgtttgtgagctttttgttccctcattctgcattaataatagctaataataaatgcatagagcaattaaactgtgaggtactgttgtatatatttttttgcctgttgattttgatgagatcttaagttactgtggatgagggtgatgcctattaagcagttgattcaaatcatattctctttaattcttaggataaaaaggttttctgctatctaaaaaaaaaaaaaaa

SEQ ID NO: 34 - DNA, 515 bp, Homo sapiens
  misc_feature: LGALS7
acggctgcccaacccggtcccagccatgtccaacgtcccccacaagtcctcactgcccgagggcatccgccctggcacggtgctgagaattcgcggcttggttcctcccaatgccagcaggttccatgtaaacctgctgtgcggggaggagcagggctccgatgccgcgctgcatttcaacccccggctggacacgtcggaggtggtcttcaacagcaaggagcaaggctcctggggccgcgaggagcgcgggccgggcgttcctttccagcgcgggcagcccttcgaggtgctcatcatcgcgtcagacgacggcttcaaggccgtggttggggacgcccagtaccaccacttccgccaccgcctgccgctggcgcgcgtgcgcctggtggaggtgggcggggacgtgcagctggactccgtgaggatcttctgagcagaagcccaggcgggcccggggccttggctggcaaataaagcgttagcccgcagcgaaaaaaaaaaaaaaaaaaaaa

(19) United States
(12) Patent Application Publication
(10) Pub. No.: US 2018/0188258 A1
(43) Pub. Date: Jul. 5, 2018

(54) MARKER FOR HETEROGENEITY OF CANCER TISSUE, AND USE THEREOF

(71) Applicant: Keio University, Tokyo (JP)

(72) Inventors: Hideyuki Saya, Tokyo (JP); Yoshimi Arima, Tokyo (JP); Takashi Semba, Tokyo (JP)

(73) Assignee: Keio University, Tokyo (JP)

(21) Appl. No.: 15/740,214

(22) PCT Filed: Jun. 30, 2016
(86) PCT No.: PCT/JP2016/069529

§ 371 Date: Dec. 27, 2017

(30) Foreign Application Priority Data
Jul. 1, 2015 (JP) 2015-133033

(51) Int. Cl. G01N 33/574; C12N 15/10; G01N 33/50

(52) U.S. Cl. CPC G01N 33/57484; G01N 2033/57403; G01N 33/5023; C12N 15/1096

(57) ABSTRACT

A genetic marker is provided that can determine whether or not a cancer tissue contains a heterogeneous cancer cell population, the genetic marker being selected from the group consisting of CALML3, BGN, CLCA2, CSTA, ALDH1A1, NGFR, S100A8, ELN, SNURF, and LGALS7.

### TECHNICAL FIELD

The present invention relates to a marker for heterogeneity of a cancer tissue and a use thereof. More specifically, the present invention relates to a genetic marker for determining whether or not a cancer tissue contains a heterogeneous cancer cell population, a protein marker for determining whether or not a cancer tissue contains a heterogeneous cancer cell population, a kit for determining whether or not a cancer tissue contains a heterogeneous cancer cell population, and a method for determining whether or not a cancer tissue sample contains a heterogeneous cancer cell population. Priority is claimed on Japanese Patent Application No. 2015-133033, filed on Jul. 1, 2015, the content of which is incorporated herein by reference.

### BACKGROUND ART

Cancer cells having different properties are known to be mixed in one cancer tissue. Such a state is called intratumor heterogeneity and is thought to be one factor that contributes to making cancer treatment difficult (see, for example, NPL 1).

### CITATION LIST

### Non-Patent Literature

[NPL 1]

Inda M. M., et al., Tumor heterogeneity is an active process maintained by a mutant EGFR-induced cytokine circuit in glioblastoma., Genes Dev., 24 (16), pp 1731-1745, 2010.

### SUMMARY OF INVENTION

### Technical Problem

Conventionally, there has not been known a method for determining heterogeneity of a cancer tissue (a state in which the cancer tissue contains a heterogeneous cancer cell population). Therefore, it is an object of the present invention to provide a genetic marker for determining whether or not a cancer tissue contains a heterogeneous cancer cell population. It is another object of the present invention to provide a protein marker for determining whether or not a cancer tissue contains a heterogeneous cancer cell population, a kit for determining whether or not a cancer tissue contains a heterogeneous cancer cell population, and a method for determining whether or not a cancer tissue contains a heterogeneous cancer cell population.

### Solution to Problem

The present invention is as follows.

(1) A genetic marker for determining whether or not a cancer tissue contains a heterogeneous cancer cell population, the genetic marker being selected from the group consisting of Calmodulin-like protein 3 (CALML3), Biglycan (BGN), Chloride channel accessory 2 (CLCA2), Cystatin A (CSTA), Aldehyde dehydrogenase 1 family, member A1 (ALDH1A1), Nerve growth factor receptor (NGFR), S100-calcium-binding protein A8 (S100A8), Elastin (ELN), SNRPN upstream reading frame (SNURF), and Galectin-7 (LGALS7).

(2) A protein marker for determining whether or not a cancer tissue contains a heterogeneous cancer cell population, the protein marker being encoded by a gene selected from the group consisting of CALML3, BGN, CLCA2, CSTA, ALDH1A1, NGFR, S100A8, ELN, SNURF, and LGALS7.

(3) A marker for determining a prognosis of cancer, including a gene selected from the group consisting of CALML3, BGN, CLCA2, CSTA, ALDH1A1, NGFR, S100A8, ELN, SNURF, and LGALS7, or a protein encoded by the gene.

(4) A kit for determining whether or not a cancer tissue contains a heterogeneous cancer cell population, including a primer set for amplifying cDNA of at least one gene selected from the group consisting of CALML3, BGN, CLCA2, CSTA, ALDH1A1, NGFR, S100A8, ELN, SNURF, and LGALS7, a probe that specifically hybridizes to mRNA of at least one gene selected from the group consisting of CALML3, BGN, CLCA2, CSTA, ALDH1A1, NGFR, S100A8, ELN, SNURF, and LGALS7, or a specific binding substance to a protein encoded by at least one gene selected from the group consisting of CALML3, BGN, CLCA2, CSTA, ALDH1A1, NGFR, S100A8, ELN, SNURF, and LGALS7.

(5) A kit for determining a prognosis of cancer, including a primer set for amplifying cDNA of at least one gene selected from the group consisting of CALML3, BGN, CLCA2, CSTA, ALDH1A1, NGFR, S100A8, ELN, SNURF, and LGALS7, a probe that specifically hybridizes to mRNA of at least one gene selected from the group consisting of CALML3, BGN, CLCA2, CSTA, ALDH1A1, NGFR, S100A8, ELN, SNURF, and LGALS7, or a specific binding substance to a protein encoded by at least one gene selected from the group consisting of CALML3, BGN, CLCA2, CSTA, ALDH1A1, NGFR, S100A8, ELN, SNURF, and LGALS7.

(6) A method for determining whether or not a cancer tissue sample contains a heterogeneous cancer cell population, including a detection step of detecting the expression of at least one gene selected from the group consisting of CALML3, BGN, CLCA2, CSTA, ALDH1A1, NGFR, S100A8, ELN, SNURF, and LGALS7 in a cancer tissue sample; and a step of determining that the cancer tissue sample contains a heterogeneous cancer cell population in the case where the expression of the gene is detected.

(7) The determination method according to (6), in which the detection step is carried out by detecting mRNA of the gene.

(8) The determination method according to (6), in which the detection step is carried out by detecting a protein encoded by the gene.

(9) The determination method according to any one of (6) to (8), in which the cancer tissue sample is derived from breast cancer, melanoma, lung cancer, or pancreatic cancer.

(10) The determination method according to (9), in which the breast cancer is an estrogen receptor(−) progesterone receptor(−) HER2(−) breast cancer.

(11) The determination method according to any one of (6) to (10), in which the heterogeneous cancer cell population includes ZEB1(+) CLDN1(−) cells and ZEB1(−) CLDN1(+) cells.

(12) A method for determining a prognosis of cancer, including a detection step of detecting the expression of at least one gene selected from the group consisting of CALML3, BGN, CLCA2, CSTA, ALDH1A1, NGFR, S100A8, ELN, SNURF, and LGALS7 in a cancer tissue sample; and a step of determining that a patient from whom the cancer tissue sample is derived has a poor prognosis in the case where the expression of the gene is detected.

(13) A heterogeneous cancer cell population expressing a gene selected from the group consisting of CALML3, BGN, CLCA2, CSTA, ALDH1A1, NGFR, S100A8, ELN, SNURF, and LGALS7.

### Advantageous Effects of Invention

According to the present invention, it is possible to provide a genetic marker and a cDNA marker for determining whether or not a cancer tissue contains a heterogeneous cancer cell population. Further, it is possible to provide a protein marker for determining whether or not a cancer tissue contains a heterogeneous cancer cell population, a kit for determining whether or not a cancer tissue contains a heterogeneous cancer cell population, and a method for determining whether or not a cancer tissue contains a heterogeneous cancer cell population.

### BRIEF DESCRIPTION OF DRAWINGS

FIG. 1(a) is a graph showing the analysis results of ZEB1 gene expression in a plurality of breast cancer cell lines. FIG. 1(b) is a graph showing the analysis results of CLDN1 gene expression in a plurality of breast cancer cell lines.

FIG. 2 is a graph showing the results of Experimental Example 3.

FIG. 3(a) is a photograph showing the results of staining a thin-sectioned tissue sample of a cancer tissue with an anti-ZEB1 antibody. FIG. 3(b) is a photograph showing the results of staining a thin-sectioned tissue sample having approximately the same field of view as FIG. 3(a) with an anti-CLDN1 antibody.

FIGS. 4(a) and 4(b) are graphs showing the results of Experimental Example 6.

FIG. 5 is a graph showing the results of Experimental Example 7.

FIG. 6 is a diagram showing the analysis results of Experimental Example 8.

FIGS. 7(a) to 7(j) are photographs showing the results of Experimental Example 10.

FIGS. 8(a) to 8(j) are graphs showing the results of quantitative real-time PCR in Experimental Example 11.

### DESCRIPTION OF EMBODIMENTS

### Marker for determining whether or not Cancer Tissue contains Heterogeneous Cancer Cell Population

In one embodiment, the present invention provides a genetic marker for determining whether or not a cancer tissue contains a heterogeneous cancer cell population, the genetic marker being selected from the group consisting of Calmodulin-like protein 3 (CALML3, accession number: NM_005185, SEQ ID NO: 25), Biglycan (BGN, accession number: NM_001711, SEQ ID NO: 26), Chloride channel accessory 2 (CLCA2, accession number: NM_006536, SEQ ID NO: 27), Cystatin A (CSTA, (StefinA), accession number: NM_005213, SEQ ID NO: 28), Aldehyde dehydrogenase 1 family, member A1 (ALDH1A1, accession number: NM_000689, SEQ ID NO: 29), Nerve growth factor receptor (NGFR, accession number: NM_002507, SEQ ID NO: 30), S100-calcium-binding protein A8 (S100A8, accession number: NM_002964, SEQ ID NO: 31), Elastin (ELN, accession number: NM_000501, SEQ ID NO: 32), SNRPN upstream reading frame (SNURF, accession number: NM_005678, SEQ ID NO: 33), and Galectin-7 (LGALS7, accession number: NM_002307, SEQ ID NO: 34), and a cDNA marker which is cDNA of such a gene.

That is, the marker of the present embodiment may be a cDNA marker for determining whether or not a cancer tissue contains a heterogeneous cancer cell population, which is selected from the group consisting of CALML3 cDNA, BGN cDNA, CLCA2 cDNA, CSTA cDNA, ALDH1A1 cDNA, NGFR cDNA, S100A8 cDNA, ELN cDNA, SNURF cDNA, and LGALS7 cDNA.

As will be described later in the EXAMPLES, the present inventors could have produced unexpectedly a cancer tissue composed of heterogeneous cell populations by mixing a cell line that does not form a tumor upon single transplantation with a cell line that forms a tumor even upon single transplantation and transplanting such a cell line mixture into a nude mouse. In addition, the present invention has been completed by identifying CALML3, BGN, CLCA2, CSTA, ALDH1A1, NGFR, S100A8, ELN, SNURF, and LGALS7 as genes which are specifically expressed in a cancer tissue containing a heterogeneous cancer cell population.

Therefore, in the case where the expression of any one or more of these genes is detected in a cancer tissue, it can be determined that the cancer tissue contains a heterogeneous cancer cell population.

In addition, as will be described later in the EXAMPLES, the present inventors have found that a cancer tissue containing a heterogeneous cancer cell population tends to have a poor prognosis. Therefore, a gene selected from the group consisting of CALML3, BGN, CLCA2, CSTA, ALDH1A1, NGFR, S100A8, ELN, SNURF, and LGALS7, and a cDNA thereof can also be said to be a genetic marker for determining a prognosis of cancer.

In the case where the expression of any one or more of these genes is detected in a cancer tissue, it can be predicted that a cancer patient having such a cancer tissue has a poor prognosis.

In the present specification, the poor prognosis means that cancer cell proliferation is fast, a metastatic ability of cancer is high, a resistance of a cancer tissue to an anticancer drug is high, a survival rate of a patient is low, or the like.

The above-mentioned genetic marker may be a splicing variant of the foregoing gene by alternative splicing or the like. In addition, the above-mentioned genetic marker may be a mutant of the foregoing gene including single nucleotide polymorphism (SNP) and the like.

The marker for determining whether or not a cancer tissue contains a heterogeneous cancer cell population may be a protein encoded by the foregoing gene. That is, the marker for determination may be a protein marker.

Also, like the above-described genetic marker, the protein encoded by a gene selected from the group consisting of CALML3, BGN, CLCA2, CSTA, ALDH1A1, NGFR, S100A8, ELN, SNURF, and LGALS7 can also be said to be a protein marker for determining a prognosis of cancer.

The expression of the foregoing genetic marker or protein marker in a cancer tissue can be detected by RT-PCR, DNA array analysis, Northern blotting, immunostaining, ELISA, Western blotting, flow cytometric analysis, or the like.

Among CALML3, BGN, CLCA2, CSTA, ALDH1A1, NGFR, S100A8, ELN, SNURF, and LGALS7 genes, cDNAs thereof, or proteins encoded by such genes, particularly CALML3, CLCA2, CSTA, and LGALS7 genes, cDNAs thereof, or proteins encoded by such genes are more preferable as the marker for determining whether or not a cancer tissue contains a heterogeneous cancer cell population. As will be described later in the EXAMPLES, the expression of CALML3, CLCA2, CSTA, and LGALS7 genes has actually been confirmed in clinical specimens of human breast cancer.

### Kit for determining whether or not Cancer Tissue contains Heterogeneous Cancer Cell Population

In one embodiment, the present invention provides a kit for determining whether or not a cancer tissue contains a heterogeneous cancer cell population, including a primer set for amplifying cDNA of at least one gene selected from the group consisting of CALML3, BGN, CLCA2, CSTA, ALDH1A1, NGFR, S100A8, ELN, SNURF, and LGALS7, a probe that specifically hybridizes to mRNA of at least one gene selected from the group consisting of CALML3, BGN, CLCA2, CSTA, ALDH1A1, NGFR, S100A8, ELN, SNURF, and LGALS7, or a specific binding substance to a protein encoded by at least one gene selected from the group consisting of CALML3, BGN, CLCA2, CSTA, ALDH1A1, NGFR, S100A8, ELN, SNURF, and LGALS7.

As described above, in the case where the expression of any one or more of the foregoing genes is detected in a cancer tissue, it can be predicted that a cancer patient having such a cancer tissue has a poor prognosis. Therefore, the kit of the present embodiment can also be said to be a kit for determining a prognosis of cancer.

(Primer Set)

The kit of the present embodiment may include a primer set for amplifying cDNA of at least one gene selected from the group consisting of CALML3, BGN, CLCA2, CSTA, ALDH1A1, NGFR, S100A8, ELN, SNURF, and LGALS7.

The sequence of the primer set is not particularly limited as long as it can amplify at least a part of cDNA of these genes. Specific examples of the base sequence of the primer set include those described later in the EXAMPLES.

At least one of the primers constituting the primer set may have a base sequence including an exon-exon boundary in the base sequence of any one of the foregoing genes. Such a primer has a base sequence which does not exist in nature.

(Probe)

The kit of the present embodiment may include a probe that specifically hybridizes to mRNA of at least one gene selected from the group consisting of CALML3, BGN, CLCA2, CSTA, ALDH1A1, NGFR, S100A8, ELN, SNURF, and LGALS7.

The probe may be, for example, a nucleic acid fragment having a base sequence complementary to the base sequence of at least a part of the mRNA of the foregoing gene. Further, the probe may have various chemical modifications for the purpose of improving the stability, specificity at the time of hybridization, and the like. For example, a phosphate residue may be substituted with a chemically modified phosphate residue such as phosphorothioate (PS), methylphosphonate, or phosphorodithionate in order to suppress degradation by a hydrolytic enzyme such as nuclease. Also, at least a part of the probe may be constituted of a nucleic acid analog such as peptide nucleic acid (PNA).

In addition, the probe may have a base sequence including an exon-exon boundary in the base sequence of any one of the foregoing genes. Such a probe has a base sequence which does not exist in nature.

The probe may be fixed on a solid phase. Examples of the solid phase include beads, plate-like substrates, membranes, and the like.

For example, the probe may be fixed to the surface of a plate-like substrate to form a microarray. In this case, for example, the expression of the foregoing gene in a cancer tissue can be detected by extracting RNA from a cancer tissue sample, labeling the extracted RNA with a fluorescent substance, hybridizing the labeled extracted RNA with a microarray, and detecting the RNA bound to the probe on the microarray. In the case where the expression of any one or more of the foregoing genes is detected, it can be determined that the cancer tissue contains a heterogeneous cancer cell population.

(Specific Binding Substance)

The kit of the present embodiment may include a specific binding substance to a protein encoded by at least one gene selected from the group consisting of CALML3, BGN, CLCA2, CSTA, ALDH1A1, NGFR, S100A8, ELN, SNURF, and LGALS7.

Examples of the specific binding substance include an antibody, an antibody fragment, and an aptamer. The antibody can be constructed, for example, by immunizing an animal such as a mouse with the foregoing protein as an antigen. Alternatively, the antibody can be constructed by screening an antibody library such as a phage library. Examples of the antibody fragment include Fv, Fab, and scFv. The antibody or antibody fragment may be polyclonal or monoclonal.

The aptamer is a substance having a specific binding ability to a labeling substance. Examples of the aptamer include a nucleic acid aptamer and a peptide aptamer. The nucleic acid aptamer having a specific binding ability to the foregoing protein may be selected by, for example, systematic evolution of ligand by exponential enrichment (SELEX). Further, the peptide aptamer having a specific binding ability to the foregoing protein may be selected by, for example, a two-hybrid method using yeast.

The specific binding substance is not particularly limited as long as it can specifically bind to the foregoing protein, and may be a commercially available product.

For example, the presence of the foregoing protein can be detected by immunostaining the thin-sectioned sample of the fixed cancer tissue using the foregoing specific binding substance. The detection of the presence of the foregoing protein may be carried out by Western blotting, ELISA, flow cytometric analysis, or the like.

The presence of any one or more of the foregoing proteins indicates that a cancer tissue contains a heterogeneous cancer cell population.

### Method for determining whether Cancer Tissue Sample contains Heterogeneous Cancer Cell Population

In one embodiment, the present invention provides a method for determining whether or not a cancer tissue sample contains a heterogeneous cancer cell population, including a detection step of detecting the expression of at least one gene selected from the group consisting of CALML3, BGN, CLCA2, CSTA, ALDH1A1, NGFR, S100A8, ELN, SNURF, and LGALS7 in a cancer tissue sample; and a step of determining that the cancer tissue sample contains a heterogeneous cancer cell population in the case where the expression of the gene is detected.

As described above, in the case where the expression of any one or more of the foregoing genes is detected in a cancer tissue sample, it can be predicted that a cancer patient having such a cancer tissue has a poor prognosis. Therefore, the determination method of the present embodiment can also be said to be a method for determining a prognosis of cancer.

The above detection step may be carried out by detecting mRNA of the foregoing gene. Alternatively, the detection step may be carried out by detecting a protein encoded by the foregoing gene.

The detection of mRNA of the foregoing gene can be carried out by RT-PCR, DNA array analysis, Northern blotting, or the like. In addition, the detection of the protein encoded by the foregoing gene can be carried out by immunostaining, ELISA, Western blotting, flow cytometric analysis, or the like.

In the case where the expression of any one or more of the foregoing genes is detected, it can be determined that the cancer tissue sample contains a heterogeneous cancer cell population.

The cancer tissue sample may be a sample derived from breast cancer, melanoma, lung cancer, or pancreatic cancer. Some of these cancers contain heterogeneous cancer cell populations, and those with heterogeneous cancer cell populations are known to have a poor prognosis.

The breast cancer may be an estrogen receptor(−) progesterone receptor(−) HER2(−) breast cancer, that is, a breast cancer that does not express an estrogen receptor, a progesterone receptor, and HER2. Such a breast cancer is also referred to as a triple negative breast cancer and it is known to be a breast cancer having a poor prognosis.

In addition, the heterogeneous cancer cell population may be a cell population containing ZEB1(+) CLDN1(−) cells, that is, cells expressing ZEB1 and not expressing CLDN1, and ZEB1(−) CLDN1(+) cells, that is, cells not expressing ZEB1 and expressing CLDN1.

Zinc finger E-box-binding homeobox 1 (ZEB1) is a transcription factor that induces epithelial-mesenchymal transition which is a phenomenon in which an epithelial cell loses its cell polarity or a cell adhesion function to the surrounding cells and obtains a migration and invasion ability so that the epithelial cell changes into a mesenchymal-like cell. Further, Claudin 1 (CLDN1) is a major protein present in a tight junction.

As will be described later in the EXAMPLES, a cancer tissue containing ZEB1(+) CLDN1(−) cells and ZEB1(−) CLDN1(+) cells can be mentioned as an example of the cancer tissue containing a heterogeneous cancer cell population.

### Heterogeneous Cancer Cell Population

In one embodiment, the present invention provides a heterogeneous cancer cell population which expresses a gene selected from the group consisting of CALML3, BGN, CLCA2, CSTA, ALDH1A1, NGFR, S100A8, ELN, SNURF, and LGALS7. The cancer cell population of the present embodiment is preferably formed in vivo. As will be described later in the EXAMPLES, it is possible to form a heterogeneous cancer cell population as a cancer tissue of a tumor-bearing mouse, for example, by transplanting a mixture of ZEB1(+) CLDN1(−) cancer cells and ZEB1(−) CLDN1(+) cancer cells into an immunodeficient mouse.

### Other Embodiments

In one embodiment, the present invention provides a cDNA of a gene selected from the group consisting of CALML3, BGN, CLCA2, CSTA, ALDH1A1, NGFR, S100A8, ELN, SNURF, and LGALS7. As described above, such a cDNA can be used as a genetic marker for determining whether or not a cancer tissue contains a heterogeneous cancer cell population, or as a marker for determining a prognosis of cancer.

In one embodiment, the present invention provides a method for detecting the expression of a gene in a cancer tissue sample, including a step of collecting the cancer tissue sample from a patient; and a step of detecting the expression of a gene selected from the group consisting of CALML3, BGN, CLCA2, CSTA, ALDH1A1, NGFR, S100A8, ELN, SNURF, and LGALS7 or a cDNA thereof in the cancer tissue sample, by polymerase chain reaction (PCR), gene expression analysis using a microarray, or detection of binding with a specific binding substance to a protein encoded by the gene.

In one embodiment, the present invention provides a method for determining whether or not a cancer tissue sample contains a heterogeneous cancer cell population, including a step of collecting the cancer tissue sample from a patient; and a step of detecting the expression of a gene selected from the group consisting of CALML3, BGN, CLCA2, CSTA, ALDH1A1, NGFR, S100A8, ELN, SNURF, and LGALS7 or a cDNA thereof in the cancer tissue sample, by polymerase chain reaction (PCR), gene expression analysis using a microarray, or detection of binding with a specific binding substance to a protein encoded by the gene. The determination method of the present embodiment can also be said to be a method for determining a prognosis of cancer.

### EXAMPLES

Hereinafter, the present invention will be described in more detail with reference to the following Examples, but the present invention is not limited thereto.

<Construction of Model Mouse having Heterogeneous Cancer Tissue>

### Experimental Example 1

(Study on Expression of ZEB1 and CLDN1 in Breast Cancer Cell Lines)

The present inventors have studied the expression of ZEB1 gene and CLDN1 gene in triple negative breast cancer cell lines, MDA-MB-436, BT549, MDA-MB-157, MDA-MB-231, Hs578T, HCC1937, BT20, and MDA-MB-468 cells by quantitative real-time PCR. Primers ZEB1 Fw (SEQ ID NO: 1) and ZEB1 Rv (SEQ ID NO: 2) were used for amplification of the ZEB1 gene. Primers CLDN-s (SEQ ID NO: 3) and CLDN-as (SEQ ID NO: 4) were used for amplification of the CLDN1 gene.

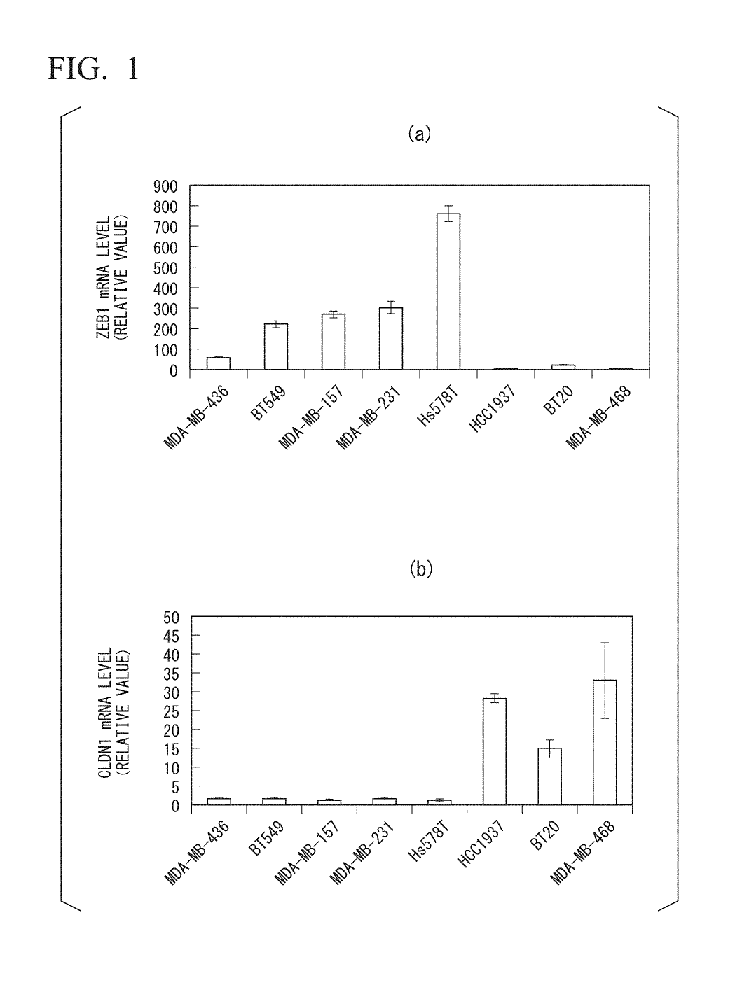

FIG. 1(a) is a graph showing the analysis results of the expression of ZEB1 gene in each breast cancer cell line. In addition, FIG. 1(b) is a graph showing the analysis results of the expression of CLDN1 gene in each breast cancer cell line.

As a result, the present inventors have found that the MDA-MB-231 cell line was ZEB1(+) CLDN1(−). The present inventors have also found that the HCC1937 cell line was ZEB1(−) CLDN1(+).

### Experimental Example 2

(Construction of Tumor-Bearing Mouse)

The MDA-MB-231 cell line and the HCC1937 cell line were mixed so that the cell number of the HCC1937 cell line was larger than the cell number of the MDA-MB-231 cell line, preferably the cell number of HCC1937 cell line:cell number of MDA-231 cell line was a ratio of 2:1 or more, for example, 9:1. Then, the cell mixture was transplanted into the fourth mammary gland adipose tissue of an immunodeficient mouse (4 weeks old, female, BALB/c-nu) to thereby construct a tumor-bearing mouse (hereinafter, sometimes referred to as “Mix mouse”). Only the MDA-MB-231 cell line and only the HCC1937 cell line were respectively transplanted into the fourth mammary gland adipose tissue of an immunodeficient mouse (4 weeks old, female, BALB/c-nu) to thereby construct tumor-bearing mice (hereinafter, sometimes referred to as “231 mouse” and “1937 mouse”, respectively) as controls.

### Experimental Example 3

(Observation of Temporal Changes in Tumor Volume)

The tumor diameters in the Mix mouse, 231 mouse, and 1937 mouse constructed in Experimental Example 2 were measured over time of about 30 days after the transplantation of the cancer cells, and temporal changes in tumor volume were observed.

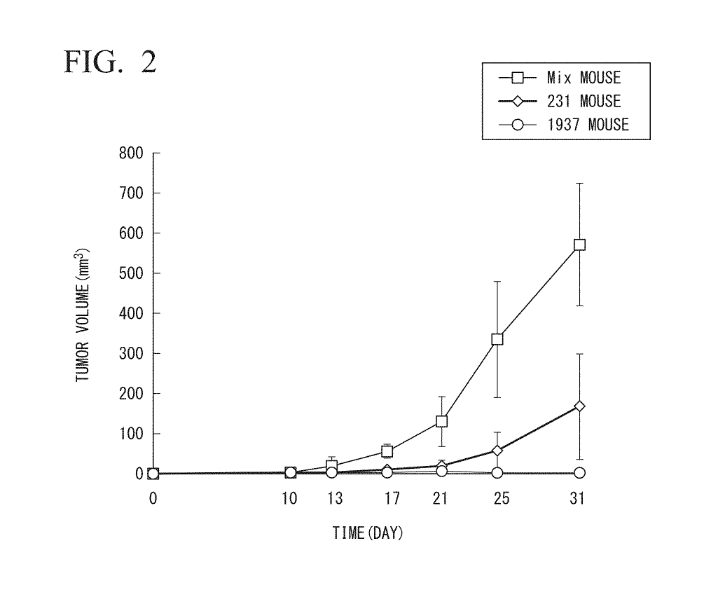

FIG. 2 is a graph showing the temporal changes in tumor volume in each tumor-bearing mouse. As a result, it was found that no tumor was formed in the 1937 mouse into which only the HCC1937 cell line had been transplanted. In addition, a tumor was formed in the Mix mouse into which the MDA-MB-231 cell line had been transplanted in admixture with the HCC1937 cell line, although a tumor was not formed in the 1937 mouse. Further, it was found that a larger tumor was formed in the Mix mouse than the 231 mouse into which only the same number of MDA-MB-231 cell line had been transplanted.

### Experimental Example 4

(Immunostaining of Cancer Tissue of Tumor-Bearing Mouse)

From the Mix mouse constructed in the same manner as in Experimental Example 2, a cancer tissue was excised 16 days after the transplantation of cancer cell lines. Subsequently, the excised cancer tissue was fixed with paraformaldehyde and embedded in paraffin. Subsequently, thin-sectioned tissue samples of the cancer tissue were prepared and the expression of ZEB1 protein and CLDN1 protein was examined by immunostaining.

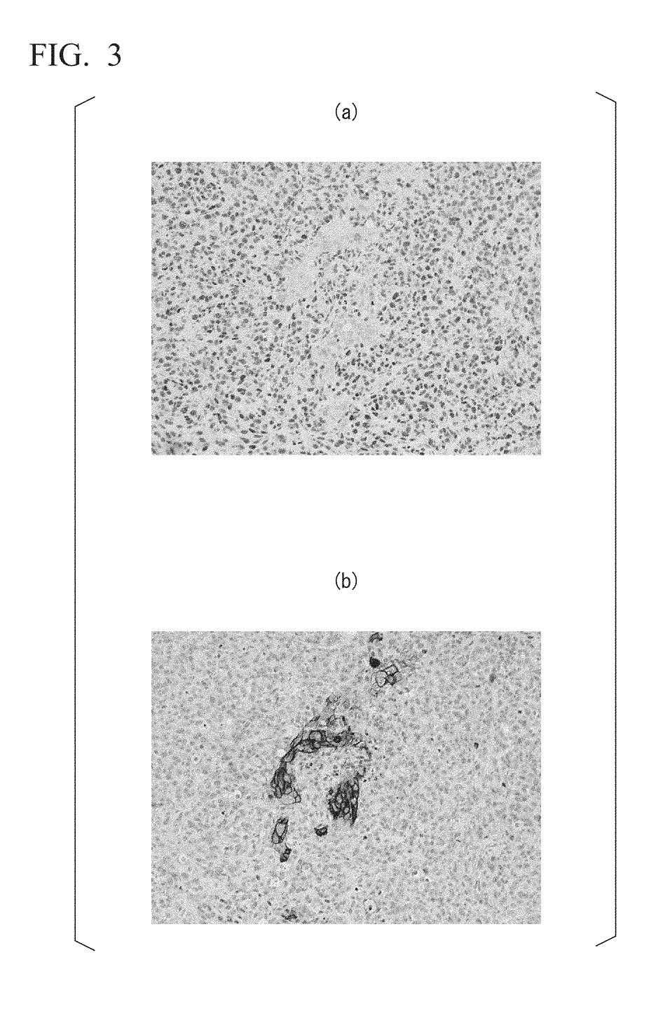

FIG. 3(a) is a photograph showing the results of staining a thin-sectioned tissue sample of a cancer tissue with an anti-ZEB1 antibody (catalog number “sc-10572”, available from Santa Cruz). FIG. 3(b) is a photograph showing the results of staining a thin-sectioned tissue sample having approximately the same field of view as FIG. 3(a) with an anti-CLDN1 antibody (catalog number “ab15098”, available from Abcam plc).

From the results of Experimental Example 3, it was found that no tumor was formed in the 1937 mouse.

From this result, it was considered that the cancer tissue of the Mix mouse is composed only of ZEB1(+) CLDN1(−) MDA-MB-231 cells and therefore the CLDN1 protein may not be expressed. On the contrary, unexpectedly, the expression of CLDN1 protein as well as ZEB1 protein was confirmed in the cancer tissue of the Mix mouse. In addition, it was shown that the expression of ZEB1 protein and the expression of CLDN1 protein were mutually exclusive, ZEB1(+) cells were CLDN1(−), and CLDN1(+) cells were ZEB1(−).

That is, it was confirmed that ZEB1(+) CLDN1(−) cells and ZEB1(−) CLDN1(+) cells were present in mixture in the cancer tissue of the Mix mouse and therefore heterogeneous cancer tissues were formed.

From the results of Experimental Examples 3 and 4, it was found that a model mouse having a heterogeneous cancer tissue can be constructed by transplanting a mixture of MDA-MB-231 cell line and HCC1937 cell line into an immunodeficient mouse. In addition, since the heterogeneous cancer tissue formed a larger tumor, it was shown that the heterogeneous cancer tissue had higher malignancy than the homogeneous cancer tissue.

<Evaluation of Malignancy of Heterogeneous Cancer Tissue>

### Experimental Example 5

(Evaluation of Lung Metastasis)

From the Mix mouse and the 231 mouse constructed in Experimental Example 2, lungs were excised 11 weeks after the transplantation of cancer cell lines, fixed with paraformaldehyde, and embedded in paraffin. Subsequently, thin-sectioned tissue samples of the lungs were prepared, stained with hematoxylin/eosin, and observed under a microscope to examine the presence of lung metastatic lesions of cancer.

As a result, there was more formation of lung metastatic lesions in the Mix mouse than the 231 mouse.

This result also indicated that the heterogeneous cancer tissue was more malignant than the homogeneous cancer tissue.

### Experimental Example 6

(Study on Therapeutic Effects by Anticancer Drug)

Paclitaxel (3 mg/kg) was administered intraperitoneally once a week to Mix mouse and 231 mouse constructed in the same manner as in Experimental Example 2, the tumor diameter was measured with time, and temporal changes in tumor volume were observed (n=5 in each case). As controls, temporal changes in tumor volume were also observed for the groups (control groups) in which physiological saline was administered intraperitoneally in place of paclitaxel (n=5 in each case).

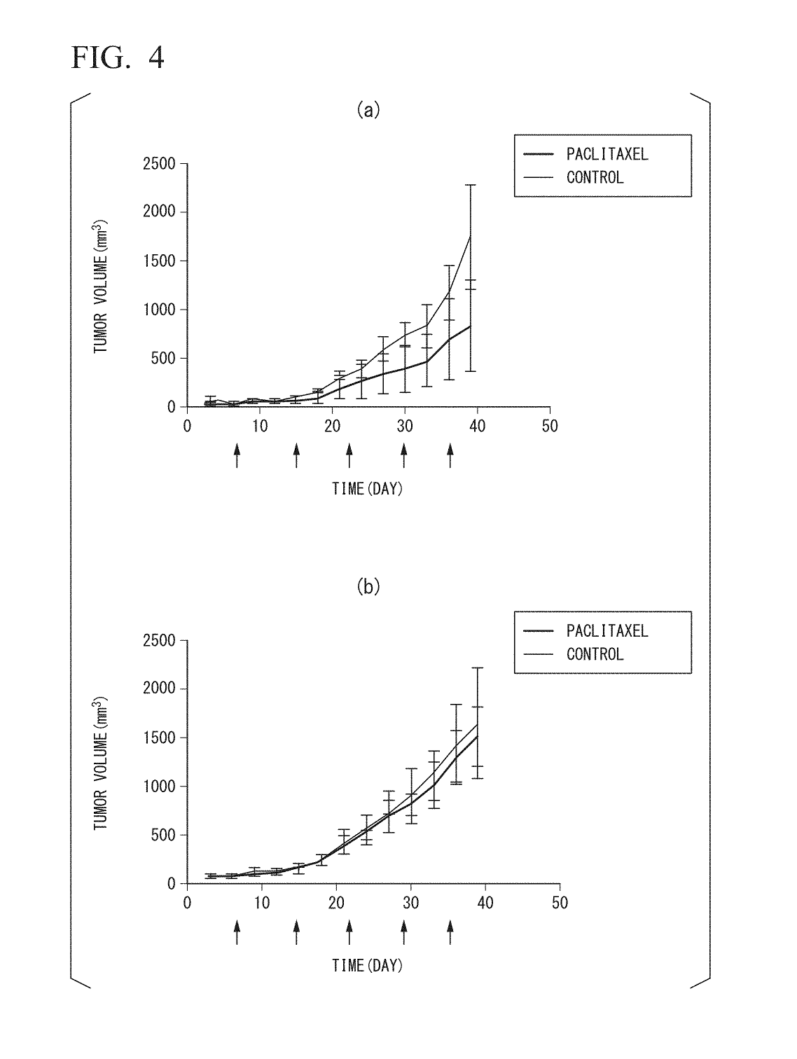

FIG. 4(a) is a graph showing the results of the 231 mouse. FIG. 4(b) is a graph showing the results of the Mix mouse. In the drawing, “Paclitaxel” indicates the results of the paclitaxel administration group and “Control” indicates the results of the control group. As a result, it was found that the cancer tissue of the Mix mouse has higher resistance to paclitaxel than the cancer tissue of the 231 mouse. This result also indicated that the heterogeneous cancer tissue was more malignant than the homogeneous cancer tissue.

### Experimental Example 7

(Study on Prognosis of ZEB1(+) CLDN1(+) Breast Cancer Patients)

Using the microarray gene expression analysis results of cancer tissue samples derived from 295 breast cancer patients (refer to Chang H. Y. et al., Robustness, scalability, and integration of a wound-response gene expression signature in predicting breast cancer survival., Proc. Natl. Acad. Sci. U.S.A., 102(10), pp 3738-3743, 2005. PMID: 15701700), the prognosis (survival rate) of ZEB1(+) CLDN1(+) breast cancer patients, ZEB1(−) CLDN1(+) breast cancer patients, ZEB1(+) CLDN1(−) breast cancer patients, and ZEB1(−) CLDN1(−) breast cancer patients was studied. Here, the cancer tissue which is ZEB1(+) CLDN1(+) was considered to be a heterogeneous cancer tissue in which ZEB1(+) CLDN1(−) cells and ZEB1(−) CLDN1(+) cells coexist.

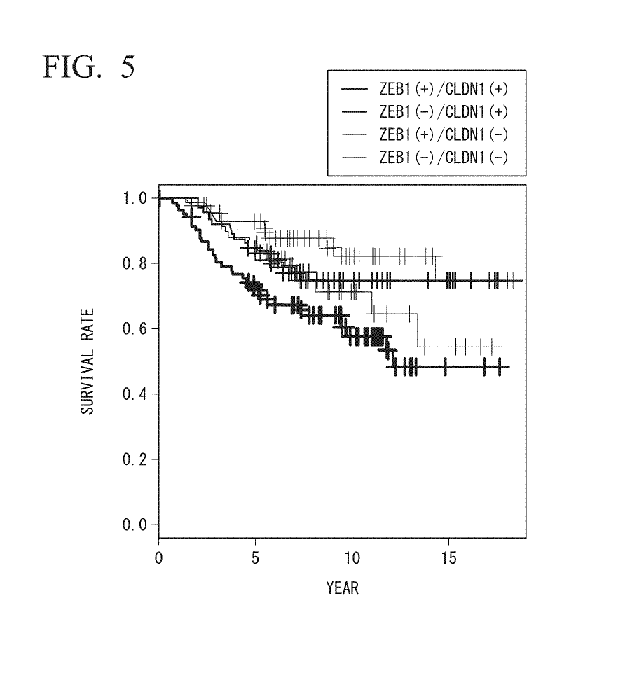

FIG. 5 is a graph showing the study results. As a result, the survival rate of ZEB1(+) CLDN1(+) breast cancer patients was found to be low. This result also indicated that the heterogeneous cancer tissue was more malignant.

As described above, the results of Experimental Examples 4 to 7 indicated that the malignancy was higher in the heterogeneous cancer tissue.

<Search for Marker for determining whether or not Cancer Tissue contains Heterogeneous Cancer Cell Population>

### Experimental Example 8

(Comprehensive Transcriptome Analysis)

The comprehensive transcriptome analysis was carried out on cancer tissues of Mix mouse, 231 mouse, and 1937 mouse constructed in the same manner as in Experimental Example 2.

First, a cancer tissue was excised from each tumor-bearing mouse and RNA was extracted therefrom. Subsequently, a library was prepared using a kit (trade name “TruSeq RNA Sample Preparation Kit v2”, available from Illumina Inc.). Subsequently, sequence analysis was carried out using a next generation sequencer (model “Genome Analyzer IIx”, available from Illumina Inc.), and bioinformatics analysis was carried out.

More specifically, the detected base sequence was separated into a human-derived base sequence and a mouse-derived base sequence using the software “Xenome”. Subsequently, the obtained base sequence data was mapped to a reference sequence using the software “Tophat”. Subsequently, the mapped base sequence data was visualized together with a known gene sequence, and mRNA expression between the respective samples was compared using the software “Avadis”.

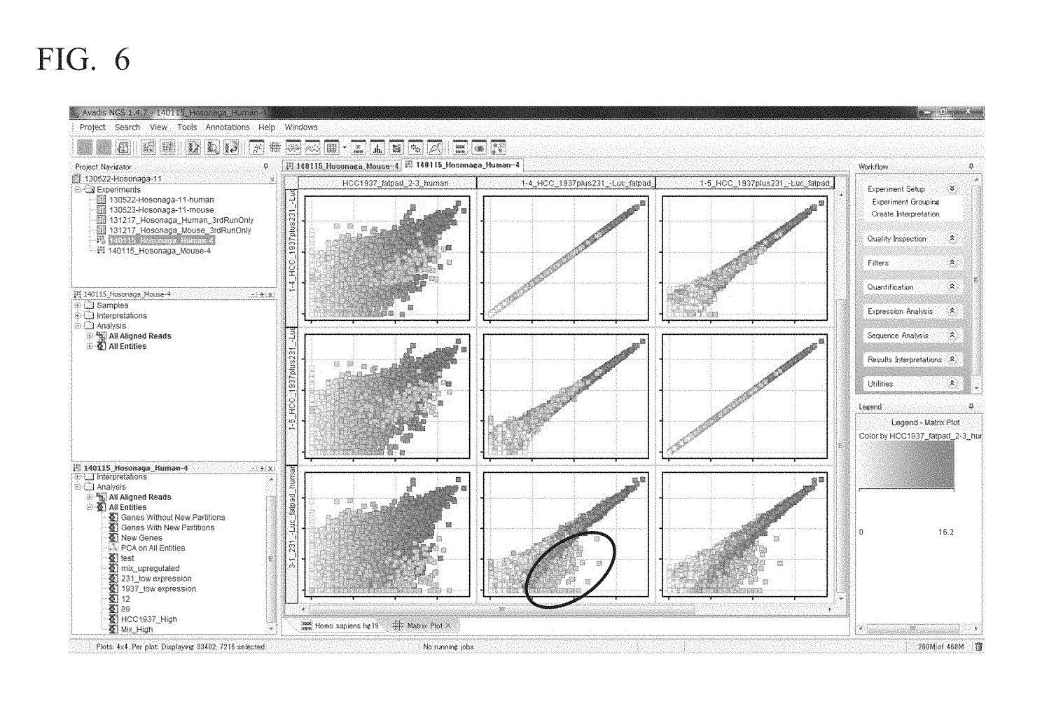

FIG. 6 is a diagram showing the analysis results using the software “Avadis”. As a result of analysis using the software “Avadis”, the genes shown in the circled area in the middle lower region of FIG. 6 were found as genes that are highly expressed in the cancer tissues of the Mix mouse and are hardly expressed in the cancer tissues of 231 mouse and 1937 mouse. These genes are candidates for a genetic marker for determining whether or not a cancer tissue contains a heterogeneous cancer cell population.

### Experimental Example 9

(Expression Analysis of Candidate Genes by Quantitative Real-Time PCR)

The quantitative real-time PCR expression analysis was carried out on the genes found in Experimental Example 8 as a candidate for a genetic marker for determining whether or not a cancer tissue contains a heterogeneous cancer cell population.

First, a cancer tissue was excised from the Mix mouse, 231 mouse and 1937 mouse constructed in the same manner as in Experimental Example 2, and RNA was extracted from a part of the tissue. In addition, a part of the tissue was fixed with paraformaldehyde, embedded in paraffin, and used for the subsequent experiment.

Subsequently, cDNA was synthesized from the extracted RNA, and the expression of the candidate genes was analyzed by quantitative real-time PCR.

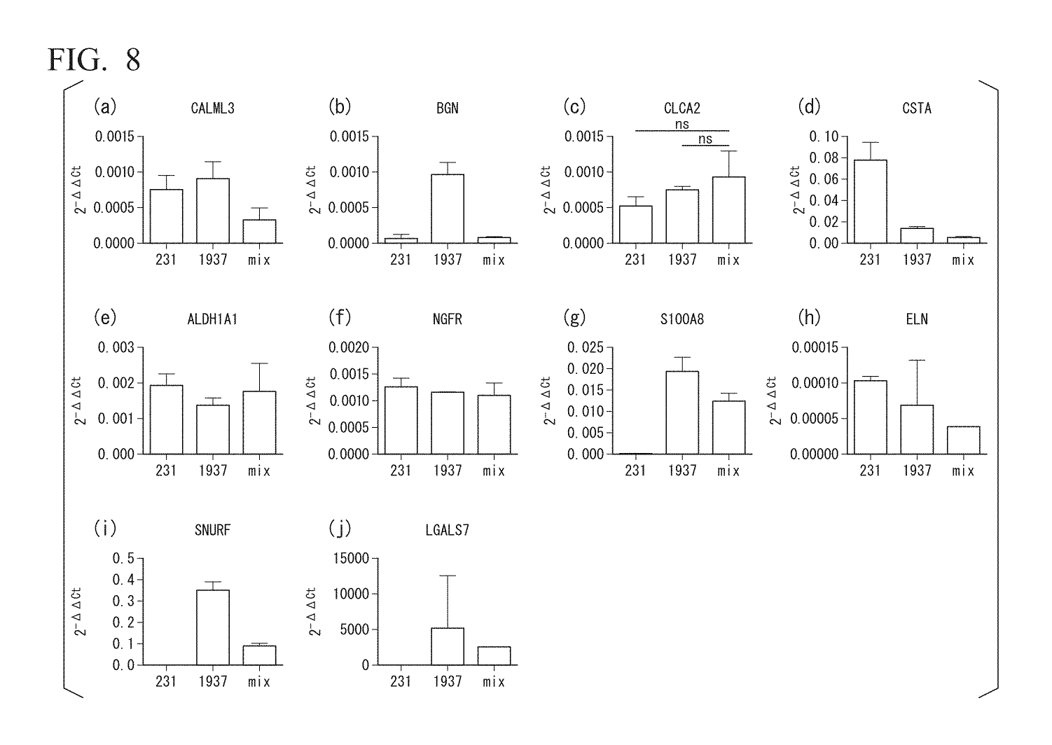

As a result, it was confirmed that CALML3, BGN, CLCA2, CSTA, ALDH1A1, NGFR, S100A8, ELN, SNURF, and LGALS7 genes, which were found to be hardly expressed in the 231 mouse and 1937 mouse in the transcriptome analysis of Example 8, were highly expressed in cancer tissues of the Mix mouse also by quantitative real-time PCR. That is, it was shown that these genes can be used as a genetic marker for determining whether or not a cancer tissue contains a heterogeneous cancer cell population, since the cancer cell population with the transplantation of mixed MDA-MB-231 cells and HCC1937 cells exhibits heterogeneity with detection of high expression of such genes. Among them, CALML3, BGN, CLCA2, CSTA, ALDH1A1, NGFR, ELN, and LGALS7 genes showed high expression in cancer tissues of the Mix mouse, despite the absence of an increase in the expression level thereof (see FIG. 8) even in the case where MDA-MB-231 cells and HCC1937 cells were mixed in in vitro experiments to be described later. In the transcriptome analysis, the expression level thereof was greater in comparison with the expression level in cancer tissues of the 231 mouse and 1937 mouse.

Table 1 shows the names and SEQ ID NOs of primers used for quantitative real-time PCR of CALML3, BGN, CLCA2, CSTA, ALDH1A1, NGFR, S100A8, ELN, SNURF, and LGALS7 genes.

TABLE 1

| | | Anti-sense |
|---|---|---|
| Gene | Sense primer (SEQ ID NO) | primer (SEQ ID NO) |
| CALML3 | CALML3-F (5) | CALML3-R (6) |
| BGN | BGN-F (7) | BGN-R (8) |
| CLCA2 | CLCA2-F (9) | CLCA2-R (10) |
| CSTA | CSTA-F (11) | CSTA-R (12) |
| ALDH1A1 | ALDH1A1-F (13) | ALDH1A1-R (14) |
| NGFR | NGFR-F (15) | NGFR-R (16) |
| S100A8 | S100A8-F (17) | S100A8-R (18) |
| ELN | ELN-F (19) | ELN-R (20) |
| SNURF | SNURF-F (21) | SNURF-R (22) |
| LGALS7 | LGALS7/7B-F (23) | LGALS7/7B-R (24) |

### Experimental Example 10

(Expression Analysis of Candidate Genes by Immunostaining)

For the genes found in Experimental Example 8 as a candidate for a genetic marker for determining whether or not a cancer tissue contains a heterogeneous cancer cell population, the expression of those genes was studied by immunostaining.

More specifically, samples of cancer tissues of the Mix mouse and 231 mouse constructed in Experimental Example 9 were thin-sectioned to prepare tissue section samples which were then subjected to hematoxylin/eosin staining and immunostaining.

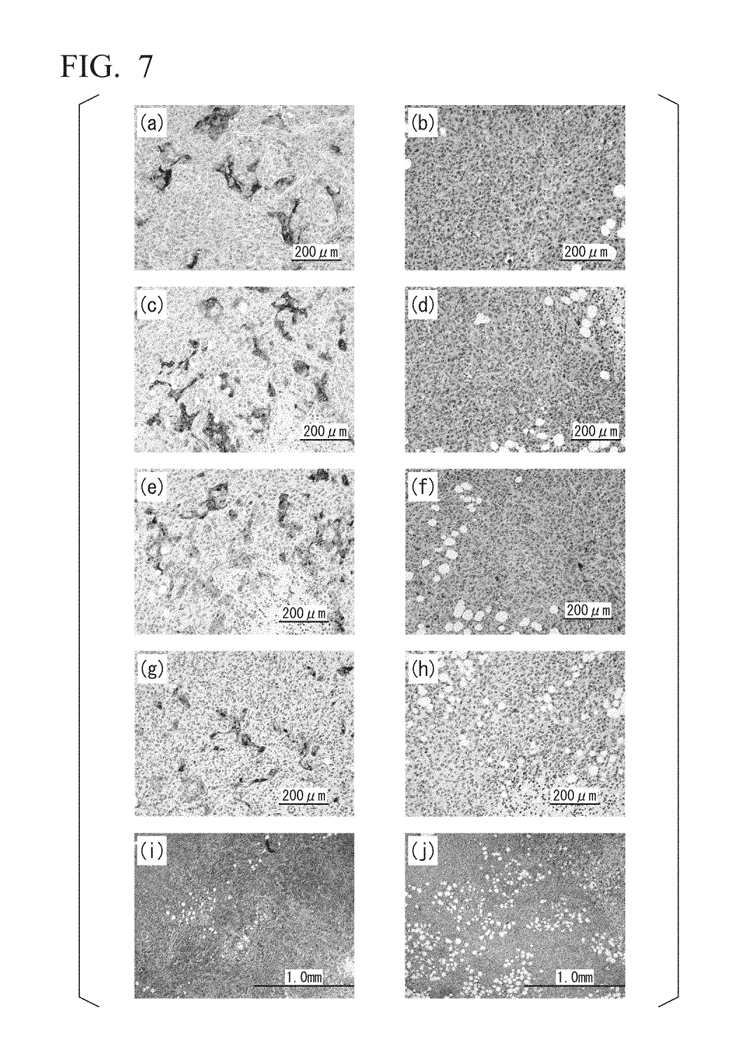

FIGS. 7(a) to 7(j) are photographs showing the representative results of immunostaining. FIG. 7(a) is a photograph showing the results of staining the cancer tissue sample of the Mix mouse with an anti-CALML3 antibody (catalog number “GTX114954”, available from Gene Tex Inc.). FIG. 7(b) is a photograph showing the results of staining the cancer tissue sample of the 231 mouse with an anti-CALML3 antibody (same as above), as a control.

FIG. 7(c) is a photograph showing the results of staining the cancer tissue sample of the Mix mouse with an anti-CLCA2 antibody (catalog number “NBP2-33482”, available from Novus Biologicals, LLC). FIG. 7(d) is a photograph showing the results of staining the cancer tissue sample of the 231 mouse with an anti-CLCA2 antibody (same as above), as a control. FIG. 7(e) is a photograph showing the results of staining the cancer tissue sample of the Mix mouse with an anti-CSTA antibody (catalog number “HPA001031”, available from ATRAS Antibodies AB). FIG. 7(f) is a photograph showing the results of staining the cancer tissue sample of the 231 mouse with an anti-CSTA antibody (same as above), as a control. FIG. 7(g) is a photograph showing the results of staining the cancer tissue sample of the Mix mouse with an anti-LGALS7 antibody (catalog number “HPA001549”, available from ATRAS Antibodies AB). FIG. 7(h) is a photograph showing the results of staining the cancer tissue sample of the 231 mouse with an anti-LGALS7 antibody (same as above), as a control. FIG. 7(i) is a photograph showing the results of staining the cancer tissue sample of the Mix mouse with an anti-S100A8 antibody (catalog number “NBP2-25269”, available from Novus Biologicals, LLC). FIG. 7(j) is a photograph showing the results of staining the cancer tissue sample of the 231 mouse with an anti-S100A8 antibody (same as above), as a control.

As a result, it was confirmed that the expression of CALML3, CLCA2, CSTA, LGALS7 and S100A8 proteins was high in cancer tissues of the Mix mouse and was hardly observed in cancer tissues of the 231 mouse.

### Experimental Example 11

(Expression Analysis of Candidate Genes by Quantitative Real-Time PCR using in Vitro Samples)

Using in vitro samples, the quantitative real-time PCR expression analysis was carried out on the genes found in Experimental Example 8 as a candidate for a genetic marker for determining whether or not a cancer tissue contains a heterogeneous cancer cell population.

First, 231 cells, 1937 cells, and cells in which 231 cells and 1937 cells were mixed in a ratio of 1:1 were respectively cultured in a culture dish for 5 days to prepare in vitro samples. Subsequently, RNA was extracted from each cell sample. Subsequently, cDNA was synthesized from the extracted RNA, and the expression of the candidate genes was analyzed by quantitative real-time PCR. The quantitative real-time PCR was carried out in the same manner as in Experimental Example 9.

FIG. 8 is a graph showing the results of quantitative real-time PCR. In FIG. 8, “231” indicates that it is a result of 231 cells, “1937” indicates that it is a result of 1937 cells, and “mix” indicates that it is a result of mixed cells of 231 cells and 1937 cells in a ratio of 1:1.

As a result, high expression of CALML3, BGN, CLCA2, CSTA, ALDHA1, NGFR, S100A8, ELN, SNURF, and LGALS7 genes as seen in in vivo samples from the transplanted cancer cell population obtained in Experimental Examples 9 and 10 could not be confirmed in the sample in which 231 cells and 1937 cells had been mixed in vitro.

From this result, it was found that a genetic marker for determining whether or not a cancer tissue contains a heterogeneous cancer cell population is not highly expressed merely by mixing and culturing cancer cells in vitro, and is highly expressed only in the case where a heterogeneous cancer cell population is formed in vivo.

### Experimental Example 12

(Immunostaining of Breast Cancer Surgical Specimens)

For the genes found in Experimental Example 8 as a candidate for a genetic marker for determining whether or not a cancer tissue contains a heterogeneous cancer cell population, the expression of such genes was studied by immunostaining of clinical specimens.

More specifically, immunohistological staining was carried out using formalin-fixed, paraffin-embedded thin-sectioned tissue samples of tissues obtained by surgery of breast cancer patients who provided informed consent, and the expression of CALML3, CLCA2, CSTA and LGALS7 proteins was studied.

As a result, staining of CALML3, CLCA2, CSTA and LGALS7 proteins was confirmed, and it was confirmed that the expression of these marker proteins could be detected in human breast cancer surgical specimens.

This result shows that genetic markers, cDNA markers, and protein markers for determining whether or not a cancer tissue contains a heterogeneous cancer cell population can be practically used in human samples.

### INDUSTRIAL APPLICABILITY

According to the present invention, it is possible to provide a genetic marker for determining whether or not a cancer tissue contains a heterogeneous cancer cell population. Further, it is possible to provide a protein marker for determining whether or not a cancer tissue contains a heterogeneous cancer cell population, a kit for determining whether or not a cancer tissue contains a heterogeneous cancer cell population, and a method for determining whether or not a cancer tissue contains a heterogeneous cancer cell population.

1. A genetic marker for determining whether or not a cancer tissue contains a heterogeneous cancer cell population, the genetic marker being selected from the group consisting of Calmodulin-like protein 3 (CALML3), Biglycan (BGN), Chloride channel accessory 2 (CLCA2), Cystatin A (CSTA), Aldehyde dehydrogenase 1 family, member A1 (ALDH1A1), Nerve growth factor receptor (NGFR), S100-calcium-binding protein A8 (S100A8), Elastin (ELN), SNRPN upstream reading frame (SNURF), and Galectin-7 (LGALS7).

2. A protein marker for determining whether or not a cancer tissue contains a heterogeneous cancer cell population, the protein marker being encoded by a gene selected from the group consisting of CALML3, BGN, CLCA2, CSTA, ALDH1A1, NGFR, S100A8, ELN, SNURF, and LGALS7.

3. A marker for determining a prognosis of cancer, comprising:
a gene selected from the group consisting of CALML3, BGN, CLCA2, CSTA, ALDH1A1, NGFR, S100A8, ELN, SNURF, and LGALS7, or a protein encoded by the gene.

4. A kit for determining whether or not a cancer tissue contains a heterogeneous cancer cell population, comprising:
a primer set for amplifying cDNA of at least one gene selected from the group consisting of CALML3, BGN, CLCA2, CSTA, ALDH1A1, NGFR, S100A8, ELN, SNURF, and LGALS7;
a probe that specifically hybridizes to mRNA of at least one gene selected from the group consisting of CALML3, BGN, CLCA2, CSTA, ALDH1A1, NGFR, S100A8, ELN, SNURF, and LGALS7; or
a specific binding substance to a protein encoded by at least one gene selected from the group consisting of CALML3, BGN, CLCA2, CSTA, ALDH1A1, NGFR, S100A8, ELN, SNURF, and LGALS7.

5. A kit for determining a prognosis of cancer, comprising:
a primer set for amplifying cDNA of at least one gene selected from the group consisting of CALML3, BGN, CLCA2, CSTA, ALDH1A1, NGFR, S100A8, ELN, SNURF, and LGALS7;
a probe that specifically hybridizes to mRNA of at least one gene selected from the group consisting of CALML3, BGN, CLCA2, CSTA, ALDH1A1, NGFR, S100A8, ELN, SNURF, and LGALS7; or
a specific binding substance to a protein encoded by at least one gene selected from the group consisting of CALML3, BGN, CLCA2, CSTA, ALDH1A1, NGFR, S100A8, ELN, SNURF, and LGALS7.

6. A method for determining whether or not a cancer tissue sample contains a heterogeneous cancer cell population, comprising:
a detection step of detecting the expression of at least one gene selected from the group consisting of CALML3, BGN, CLCA2, CSTA, ALDH1A1, NGFR, S100A8, ELN, SNURF, and LGALS7 in a cancer tissue sample; and
a step of determining that the cancer tissue sample contains a heterogeneous cancer cell population in the case where the expression of the gene is detected.

7. The determination method according to claim 6, wherein the detection step is carried out by detecting mRNA of the gene.

8. The determination method according to claim 6, wherein the detection step is carried out by detecting a protein encoded by the gene.

9. The determination method according to claim 6, wherein the cancer tissue sample is derived from breast cancer, melanoma, lung cancer, or pancreatic cancer.

10. The determination method according to claim 9, wherein the breast cancer is an estrogen receptor(−) progesterone receptor(−) HER2(−) breast cancer.

11. The determination method according to claim 6, wherein the heterogeneous cancer cell population includes ZEB1(+) CLDN1(−) cells and ZEB1(−) CLDN1(+) cells.

12. A method for determining a prognosis of cancer, comprising:
a detection step of detecting the expression of at least one gene selected from the group consisting of CALML3, BGN, CLCA2, CSTA, ALDH1A1, NGFR, S100A8, ELN, SNURF, and LGALS7 in a cancer tissue sample; and
a step of determining that a patient from whom the cancer tissue sample is derived has a poor prognosis in the case where the expression of the gene is detected.

13. A heterogeneous cancer cell population expressing a gene selected from the group consisting of CALML3, BGN, CLCA2, CSTA, ALDH1A1, NGFR, S100A8, ELN, SNURF, and LGALS7.

14. The determination method according to claim 8, wherein the cancer tissue sample is derived from breast cancer, melanoma, lung cancer, or pancreatic cancer.

15. The determination method according to claim 14, wherein the breast cancer is an estrogen receptor(−) progesterone receptor(−) HER2(−) breast cancer.

16. The determination method according to claim 8, wherein the heterogeneous cancer cell population includes ZEB1(+) CLDN1(−) cells and ZEB1(−) CLDN1(+) cells.

17. The determination method according to claim 9, wherein the heterogeneous cancer cell population includes ZEB1(+) CLDN1(−) cells and ZEB1(−) CLDN1(+) cells.

18. The determination method according to claim 10, wherein the heterogeneous cancer cell population includes ZEB1(+) CLDN1(−) cells and ZEB1(−) CLDN1(+) cells.
